(19) United States
(12) Patent Application Publication
(10) Pub. No.: US 2019/0239739 A1
(43) Pub. Date: Aug. 8, 2019

(54) ENDOSCOPE

(71) Applicant: OLYMPUS CORPORATION, Tokyo (JP)

(72) Inventor: Jin HIRAOKA, Sagamihara-shi (JP)

(73) Assignee: OLYMPUS CORPORATION, Tokyo (JP)

(21) Appl. No.: 16/388,153

(22) Filed: Apr. 18, 2019

Related U.S. Application Data
(63) Continuation of application No. PCT/JP2017/039203, filed on Oct. 30, 2017.

(30) Foreign Application Priority Data
Oct. 28, 2016 (JP) 2016-212188

(51) Int. Cl. A61B 1/12; A61B 1/018; A61B 1/00; B08B 1/00

(52) U.S. Cl. CPC A61B 1/122; A61B 1/018; B08B 1/002; A61B 1/00098; A61B 1/00087

(57) ABSTRACT

An endoscope includes an insertion portion, insertable into a subject, which allows a treatment tool to protrude from a distal end side thereof, and includes: a hard member having a treatment tool insertion hole, and a housing groove extending toward a distal end from the treatment tool insertion hole; and a raising base housed rotatably in the housing groove, to be in a raised state or an inverted state, by rotating the raising base, the raising base being abuttable on the treatment tool to adjust a protruding direction of the treatment tool from the housing groove, wherein a first introduction portion, a uniform portion, and a second introduction portion are provided continuously to form a space from a distal end to a proximal end of the insertion portion and between the raising base and a bottom of the housing groove while the raising base is in the inverted state.

### CROSS-REFERENCE TO RELATED APPLICATION

This application is a continuation of PCT International Application No. PCT/JP2017/039203 filed on Oct. 30, 2017, which designates the United States, incorporated herein by reference, and which claims the benefit of priority from Japanese Patent Application No. 2016-212188, filed on Oct. 28, 2016, incorporated herein by reference.

### BACKGROUND

The present disclosure relates to an endoscope.

In the past, an endoscope has been known which includes a flexible and elongated insertion portion that is to be inserted into a subject such as a human body to observe the inside of the subject (for example, see Japanese Laid-open Patent Publication No. 2013-183964 A (FIGS. 8 and 9), (referred to as JP 2013-183964 A hereinafter).

In the endoscope described in JP 2013-183964 A (FIGS. 8 and 9), a distal end side of the insertion portion has a distal end hard portion and a raising base. Note that the “distal end side” described below refers to a distal end side of the insertion portion. In addition, the “proximal end side” described below refers to a side opposite to the distal end of the insertion portion.

The distal end hard portion includes a treatment tool insertion tube through which a treatment tool such as a puncture needle is inserted and a hollow space that communicates with the treatment tool insertion tube and is positioned in the distal end side relative to the treatment tool insertion tube.

The raising base is rotatably housed in the hollow space of the distal end hard portion and is rotated to be set to a raised state (erected position) or an inverted state (non-erected position). With this configuration, the raising base may abut on the treatment tool inserted into the hollow space through the treatment tool insertion tube and thus adjust a protruding direction of the treatment tool from the hollow space.

### SUMMARY

The present disclosure is directed to an endoscope.

According to a first aspect of the present disclosure, an endoscope is provided which includes an insertion portion that is insertable into a subject and allows a treatment tool to protrude from a distal end side of the insertion portion, the insertion portion being cleanable with a cleaning tool having a predetermined dimension. The insertion portion includes: a hard member having a treatment tool insertion hole through which the treatment tool is allowed to be inserted, and a housing groove that communicates with the treatment tool insertion hole and extends toward a distal end from the treatment tool insertion hole; and a raising base that is housed rotatably in the housing groove, wherein the raising base is set to be in a raised state in which the raising base is raised with respect to an insertion axis along which the insertion portion is inserted into the subject and in an inverted state in which the raising base is inverted, by rotating the raising base. The raising base is abuttable on the treatment tool inserted into the housing groove through the treatment tool insertion hole so that a protruding direction of the treatment tool from the housing groove is adjusted, wherein a first introduction portion, a uniform portion, and a second introduction portion are provided continuously to form a space from a distal end side to a proximal end side of the insertion portion and between the raising base and a bottom of the housing groove while the raising base is set to be in the inverted state, the uniform portion having a first gap having a first distance smaller than the predetermined dimension of the cleaning tool between the bottom of the housing groove and the raising base, the first introduction portion having a second gap having a second distance greater than the first distance and being positioned on the distal end side with respect to the uniform portion, and the second introduction portion having a third gap having a third distance greater than the first distance and being positioned on the proximal end side with respect to the uniform portion.

According to a second aspect of the present disclosure, a method of cleaning the insertion portion of the endoscope of the first aspect is provided which includes setting the raising base to be in the inverted state; inserting the brush having the predetermined dimension into the space between the raising base and the bottom of the housing groove, the space being open on a distal end and on a proximal end thereof, from the distal end or the proximal end; and moving the brush forward and backward within the space so that the space is cleaned end to end while the brush is kept thereinside.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram schematically illustrating an endoscope system according to an embodiment of the disclosure;

FIG. 2 is a perspective view illustrating a distal end of an insertion portion;

FIG. 3 is a diagram illustrating shapes of a housing groove and a raising base;

FIG. 4 is a cross-sectional view illustrating a configuration of a rotation mechanism;

FIG. 5 is a cross-sectional view illustrating a configuration of the rotation mechanism;

FIG. 6A is a diagram illustrating an advantageous effect according to an embodiment of the disclosure; and

FIG. 6B is a diagram illustrating an advantageous effect according to an embodiment of the disclosure.

### DETAILED DESCRIPTION

Modes for embodying the present disclosure (hereinafter, referred to as “embodiment”) will now be described with reference to the accompanying drawings. Note that the present disclosure is not limited to the embodiments described below. In addition, throughout the drawings, like reference numerals denote like elements.

### Schematic Configuration of Endoscope System

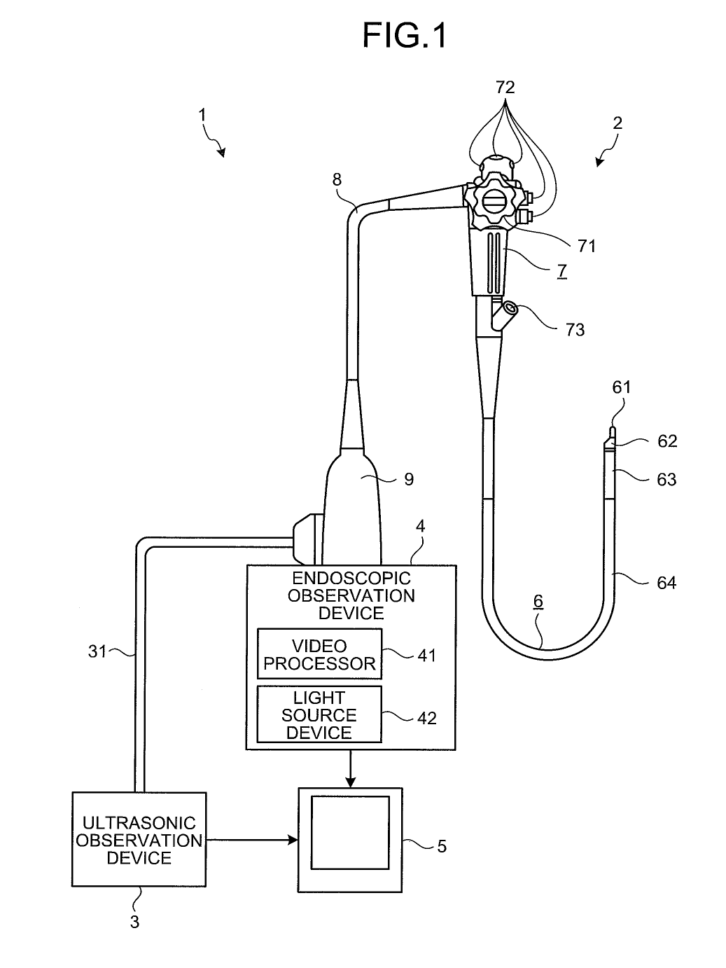

FIG. 1 is a diagram schematically illustrating an endoscope system 1 according to an embodiment of the disclosure.

The endoscope system 1 is a system of performing an ultrasonic diagnosis for the inside of a subject such as a human body using an ultrasonic endoscope. The endoscope system 1 includes an ultrasonic endoscope 2, an ultrasonic observation device 3, an endoscopic observation device 4, and a display device 5 as illustrated in FIG. 1.

The ultrasonic endoscope 2 functions as an endoscope according to the present disclosure. The ultrasonic endoscope 2 is partially insertable into the inside of the subject and is configured to transmit ultrasonic pulses to a body wall of the inside of the subject and receiving an ultrasonic echo reflected on the subject to output the echo signal. The ultrasonic endoscope 2 is also configured to capture an image of the inside of the subject, and to output an image signal.

The configuration of the ultrasonic endoscope 2 will be described in detail later.

The ultrasonic observation device 3 is electrically connected to the ultrasonic endoscope 2 through an ultrasonic cable 31 (FIG. 1). The ultrasonic observation device 3 is configured to output the pulse signal to the ultrasonic endoscope 2 through the ultrasonic cable 31 and to input the echo signal from the ultrasonic endoscope 2. In addition, the ultrasonic observation device 3 generates an ultrasonic sound image by performing a predetermined processing on the echo signal.

An endoscope connector 9 (FIG. 1) of the ultrasonic endoscope 2 is detachably connected to the endoscopic observation device 4 as described below. The endoscopic observation device 4 has a video processor 41 and a light source device 42 as illustrated in FIG. 1.

The video processor 41 receives an image signal from the ultrasonic endoscope 2 through the endoscope connector 9. In addition, the video processor 41 generates an endoscopic image by performing a predetermined processing on the image signal.

The light source device 42 supplies illumination light for illuminating the inside of the subject to the ultrasonic endoscope 2 through the endoscope connector 9.

The display device 5 includes a liquid crystal or organic electroluminescence (EL) element to display the ultrasonic sound image generated by the ultrasonic observation device 3 or the endoscopic image generated by the endoscopic observation device 4.

### Configuration of Ultrasonic Endoscope

The ultrasonic endoscope 2 includes an insertion portion 6, an operating unit 7, a universal cord 8, and the endoscope connector 9 as illustrated in FIG. 1.

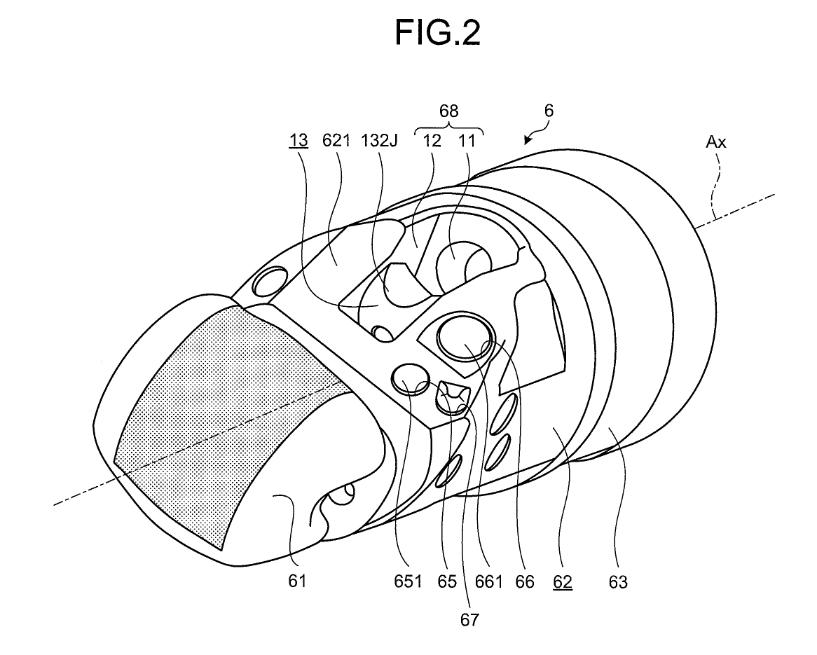

FIG. 2 is a perspective view illustrating a distal end part of the insertion portion 6.

Note that the “distal end side” described below refers to a distal end side (distal end side in an insertion direction to the inside of the subject) of the insertion portion 6. In addition, the “proximal end side” described below refers to a side opposite to the distal end of the insertion portion 6.

The insertion portion 6 is to be inserted into the inside of the subject. As illustrated in FIG. 1 or 2, the insertion portion 6 includes an ultrasonic probe 61 provided in the distal end side, a hard member 62 connected to the proximal end side of the ultrasonic probe 61, a bendable portion 63 connected to the proximal end side of the hard member 62 to be bendable, and a flexible pipe 64 connected to the proximal end side of the bendable portion 63 to provide flexibility.

Here, the insertion portion 6, the operating unit 7, the universal cord 8, and the endoscope connector 9 are provided in their insides with a light guide (not illustrated) for transmitting the illumination light supplied from the light source device 42, a transducer cable (not illustrated) for transmitting the pulse signal or the echo signal described above, a signal cable (not illustrated) for transmitting the image signal, and a conduit (not illustrated) for flowing a fluid.

Configurations of the ultrasonic probe 61 and the hard member 62 out of members 61 to 64 of the insertion portion 6 will be described.

The ultrasonic probe 61, which is a convex type ultrasonic probe, has a plurality of ultrasonic transducers (not illustrated) arranged regularly to form a convex arc shape. Note that the ultrasonic probe 61 is not limited to the convex type ultrasonic probe, but a radial type ultrasonic probe may also be employed.

Here, the ultrasonic transducer has an acoustic lens, a piezoelectric element, and a matching layer to obtain an ultrasonic echo contributing an ultrasonic tomographic image for the side inner than the body wall of the subject.

The ultrasonic probe 61 converts the pulse signal input from the ultrasonic observation device 3 through the ultrasonic cable 31 and the transducer cable (not illustrated) described above into an ultrasonic pulse and transmits the ultrasonic pulse to the inside of the subject. In addition, the ultrasonic probe 61 converts the ultrasonic echo reflected from the inside of the subject into an electric echo signal and outputs the echo signal to the ultrasonic observation device 3 through the transducer cable (not illustrated) and the ultrasonic cable 31.

The hard member 62 is a member formed of a hard material such as a resin material and has a substantially circular column shape extending along an insertion axis Ax (FIG. 2). Here, the insertion axis Ax refers to an axis extending in an extension direction of the insertion portion 6.

The hard member 62 is provided on an outer peripheral surface of the distal end side with a sloped surface 621 so that the hard member 62 is tapered toward the distal end.

Referring to FIG. 2, the hard member 62 has an installation hole (not illustrated) penetrating from the proximal end to the distal end, and an illumination hole 65, a photographing hole 66, an air/water supply hole 67, a treatment tool channel 68, and the like that penetrate from the proximal end to the sloped surface 621.

The installation hole (not illustrated) is a hole for installing the ultrasonic probe 61. In addition, the aforementioned transducer cable (not illustrated) electrically connected to the ultrasonic probe 61 is inserted into the installation hole.

An emission end side of the light guide (not illustrated) described above and an illumination lens 651 (FIG. 2) for irradiating the illumination light emitted from the emission end side of the light guide into the inside of the subject are arranged in the inside of the illumination hole 65.

An objective optical system 661 (FIG. 2) that condenses light irradiated to the inside of the subject and reflected from the inside of the subject (subject image), and an image sensor (not illustrated) that captures the subject image condensed by the objective optical system 661 are arranged in the inside of the photographing hole 66. In addition, the image signal generated by the image sensor is transmitted to the endoscopic observation device 4 (video processor 41) through the signal cable (not illustrated) described above.

According to this embodiment, the illumination hole 65 and the photographing hole 66 are formed on the sloped surface 621 as described above. For this reason, the ultrasonic endoscope 2 according to this embodiment is configured as an oblique-viewing endoscope in which a subject is observed in a direction crossing the insertion axis Ax at an acute angle. In addition, the photographing hole 66, the objective optical system 661, and the image sensor (not illustrated) function as an optical observation unit according to the present disclosure.

The air/water supply hole 67 constitutes a part of the aforementioned conduit (not illustrated) as a hole for supplying the air or water to the photographing hole 66 to clean an outer surface of the objective optical system 661.

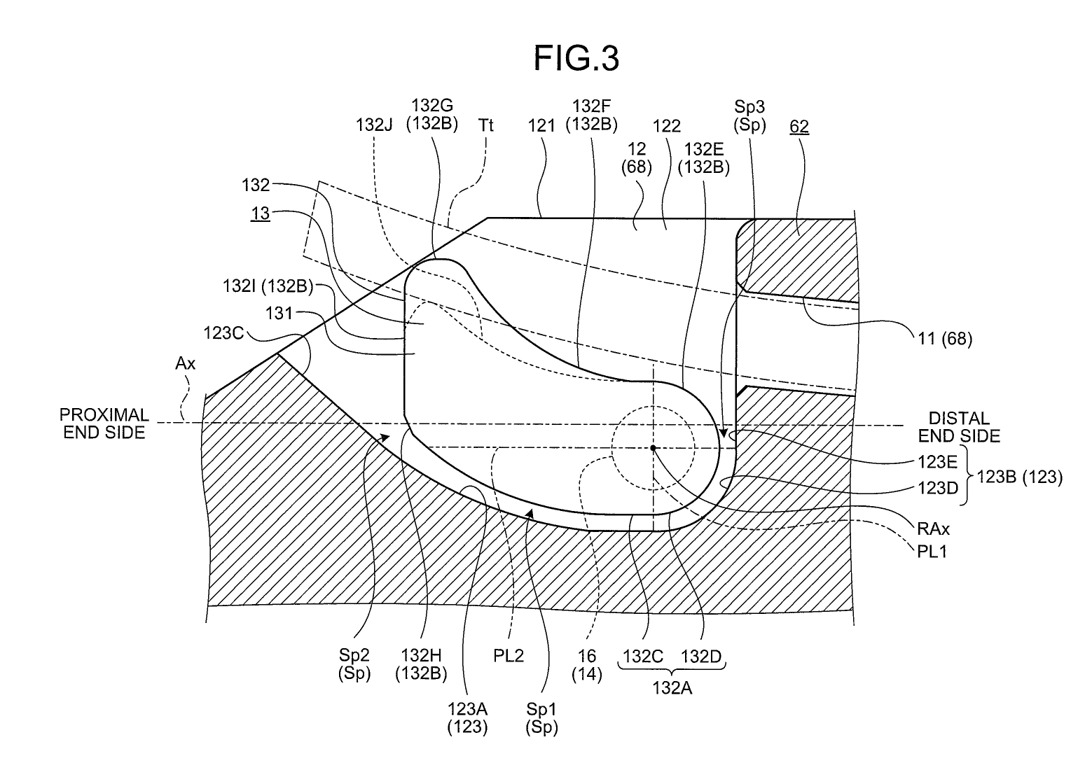

The treatment tool channel 68 is a passage that allows a treatment tool Tt (refer to FIG. 3) or a surgical tool such as a puncture needle inserted into the inside of the insertion portion 6 to protrude to the outside. As illustrated in FIG. 2, the treatment tool channel 68 has a treatment tool insertion hole 11 and a housing groove 12.

The treatment tool insertion hole 11 is a part extending from the proximal end of the hard member 62 to the distal end side to insert the treatment tool Tt.

The housing groove 12 is a groove communicating with the treatment tool insertion hole 11 and extending from the treatment tool insertion hole 11 to the distal end side along the insertion axis Ax.

The housing groove 12 houses a raising base 13 that is rotatable with respect to a rotational axis RAx (refer to FIG. 3). The raising base 13 is configured to abut on the treatment tool Tt inserted into the housing groove 12 through the treatment tool insertion hole 11 so that a protruding direction of the treatment tool Tt from the housing groove 12 is adjusted.

Note that specific shapes of the housing groove 12 and the raising base 13 will be described below.

Referring back to FIG. 1, the operating unit 7 is a part connected to the proximal end side of the insertion portion 6 for receiving various operations from a doctor or the like and functions as an operation-receiving unit according to the present disclosure. As illustrated in FIG. 1, the operating unit 7 has a bending knob 71 for bendably operating the bendable portion 63 and a plurality of operating members 72 for performing various operations.

The operating unit 7 has a treatment tool insertion opening 73 communicating with the treatment tool insertion hole 11 through a tube (not illustrated) provided in the inside of the bendable portion 63 and the flexible pipe 64. The treatment tool Tt is inserted from the treatment tool insertion opening 73 into the tube.

The universal cord 8 is a cord extending from the operating unit 7 and having a tube (not illustrated) constituting a part of the aforementioned light guide (not illustrated), the aforementioned transducer cable (not illustrated), the aforementioned signal cable (not illustrated), and the aforementioned conduit (not illustrated).

The endoscope connector 9 is provided in an end of the universal cord 8. In addition, the endoscope connector 9 is connected with the ultrasonic cable 31 and inserted into the endoscopic observation device 4 so as to electrically connect to the video processor 41 and the light source device 42.

### Shapes of Housing Groove and Raising Base

Next, shapes of the housing groove 12 and the raising base 13 will be described.

FIG. 3 is a diagram illustrating the shapes of the housing groove 12 and the raising base 13. Specifically, FIG. 3 is a cross-sectional view taken along the insertion axis Ax by cutting the hard member 62 on a cut surface passing through the housing groove 12. FIG. 3 illustrates a state in which the raising base 13 is set to an inverted state.

In the following description, an upper side of FIG. 3 (an opening 121 side of the housing groove 12) is referred to as “upward”, and a lower side of FIG. 3 will be referred to as “downward”.

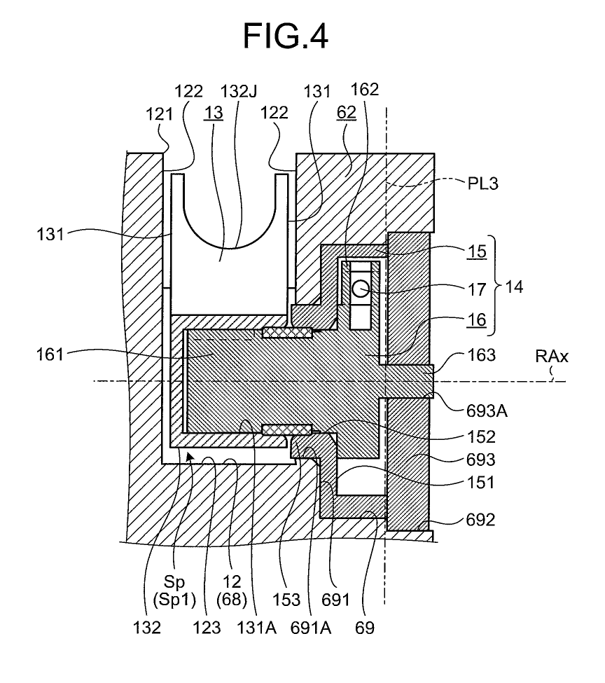

In the housing groove 12, a pair of side wall surfaces 122 are formed on flat surfaces arranged perpendicularly to the rotational axis RAx and in parallel with each other (refer to FIG. 4).

In the housing groove 12, a bottom 123 is shaped as described below. Note that, in the following description, a distal end side of the bottom 123 with respect to a first plane PL1 (FIG. 3) perpendicular to the insertion axis Ax through the rotational axis RAx will be referred to as a distal end side bottom 123A, and a proximal end side of the bottom 123 will be referred to as a proximal end side bottom 123B.

The distal end side bottom 123A has a curved surface curved upward from a boundary position with the proximal end side bottom 123B to the distal end so that the distal end serves as an edge of the opening 121. That is, the distal end side of the distal end side bottom 123A has a sloped surface 123C sloped with respect to the first plane PL1.

The proximal end side bottom 123B includes a curved surface 123D extending from the boundary position with the distal end side bottom 123A to the proximal end side to form a quarter circle centered at the rotational axis RAx, and a plane 123E extending upward from the curved surface 123D in parallel with the first plane PL1 and having an upper end serving as an edge of the opening 121. In addition, the treatment tool insertion hole 11 opens to the plane 123E and communicates with the housing groove 12.

The raising base 13 is substantially a columnar body having a columnar shape extending along the rotational axis RAx.

In the raising base 13, a pair of bottom surfaces 131 of the columnar body are perpendicular to the rotational axis RAx and form flat surfaces arranged in parallel with each other (refer to FIG. 4). A fixation hole 131A is formed which is recessed along the rotation axis RAx from one bottom surface 131 toward the other bottom surface 131 of the pair of bottom surfaces 131 (refer to FIG. 4).

In the raising base 13, the outer peripheral surface 132 of the columnar body is shaped as described below. Note that, in the following description, a lower side of the raising base 13 with respect to a second plane PL2 perpendicular to the first plane PL1 through the rotational axis RAx will be referred to as a lower outer peripheral surface 132A, and an upper side of the raising base 13 will be referred to as an upper outer peripheral surface 132B.

The lower outer peripheral surface 132A includes a first curved surface 132C curved along the distal end side bottom 123A and a second curved surface 132D extending along the curved surface 123D from the proximal end side boundary position with the first curved surface 132C. That is, the lower outer peripheral surface 132A has a shape matching with shapes of the distal end side bottom 123A and the curved surface 123D. In addition, the raising base 13 is postured such that a uniform gap is provided between the distal end side bottom 123A and the curved surface 123D and the lower outer peripheral surface 132A in the inverted state. Specifically, the raising base 13 is supported rotatably about the rotational axis RAx that is set at a position deviated toward the upper side and the distal end side with respect to the distal end side bottom 123A and the curved surface 123D by a length obtained by adding the gap and a radius of curvature of the second curved surface 132D. In other words, the raising base 13 is rotatably supported about a center of curvature of the second curved surface 132D (rotational axis RAx) so that the center of curvature of the second curved surface 132D matches with a center of curvature of the curved surface 123D. Note that the aforementioned gap corresponds to a uniform portion Sp1 (FIG. 3) according to the present disclosure.

The upper outer peripheral surface 132B includes third to sixth curved surfaces 132E to 132H and a plane 132I.

The third curved surface 132E is a curved surface that meets the lower outer peripheral surface 132A at the second plane PL2, when the raising base 13 is in the inverted state, and extends upward therefrom. Namely, the third curved surface 132E extends from a proximal end side boundary position between the lower outer peripheral surface 132A (proximal end side boundary position with the second curved surface 132D) and the third curved surface 132E to the distal end side. The third curved surface 132E forms a quarter circles having the same curvature as that of the second curved surface 132D.

The fourth curved surface 132F is a curved surface that extends continuously from the third curved surface 132E. The fourth curved surface 132F is curved upward from the distal end side boundary position between the third curved surface 132E and the fourth curved surface 132F to the distal end.

The fifth curved surface 132G is a curved surface that extends continuously from the fourth curved surface 132F and is curved in an arc shape toward the distal end side so as to protrude upward from the distal end side boundary position between the fourth curved surface 132F and the fifth curved surface 132G.

The plane 132I is a plane that extends continuously from the fifth curved surface 132G and extends downward from the distal end side boundary position between the fifth curved surface 132G and the plane 132I. The plane 132I is in parallel with the first plane PL1 while the raising base 13 is set to the inverted state.

The outer peripheral surface 132 has a recessed groove 132J (FIG. 3) having a U-shaped cross section for placing the treatment tool Tt inserted into the housing groove 12 through the treatment tool insertion hole 11. The recessed groove 132J extends from the boundary position between the third curved surface 132E and the fourth curved surface 132F through the plane 132I along the insertion axis Ax.

The sixth curved surface 132H is a curved surface for connecting the plane 132I and the lower outer peripheral surface 132A, and is curved in an arc shape toward the proximal end side so as to protrude downward with a curvature smaller than that of the distal end side bottom 123A in the lower side boundary position between the plane 132I and the sixth curved surface 132H.

In order to set the raising base 13 to the inverted state, as illustrated in FIG. 3, a gap between the sixth curved surface 132H and the distal end side bottom 123A and a gap between the third curved surface 132E and the proximal end side bottom 123B become larger than the gap of the uniform portion Sp1. Such gaps correspond to introduction portions Sp2 and Sp3 (FIG. 3), respectively, according to the present disclosure.

That is, when the raising base 13 is set to the inverted state, a space Sp (FIG. 3) including the uniform portion Sp1 and the introduction portions Sp2 and Sp3, continuously extending from the distal end side to the proximal end side, and having both ends opened in both the distal end side and the proximal end side is formed between the bottom 123 and the raising base 13.

Note that the radiuses of curvature of the second and third curved surfaces 132D and 132E and the curved surface 123D are preferably set to twice or larger and five times or smaller than the gap of the uniform portion Sp1.

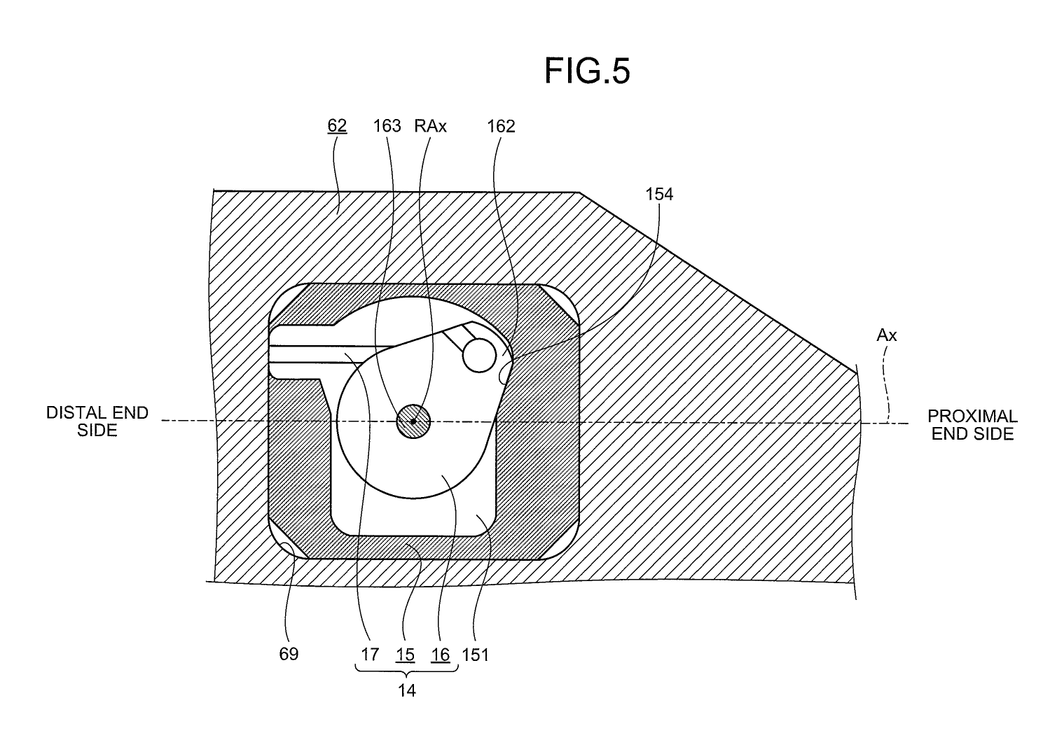

The raising base 13 described above is rotated by a rotation mechanism 14 (refer to FIGS. 4 and 5) operating in response to an operation performed by a doctor or the like on the operating unit 7.

A configuration of the rotation mechanism 14 will now be described.

### Configuration of Rotation Mechanism

FIGS. 4 and 5 are cross-sectional views illustrating a configuration of the rotation mechanism 14. Specifically, FIG. 4 is a diagram as a part of the cross section taken by cutting the hard member 62 on a cut surface passing through the first plane PL1 is seen from the proximal end side. FIG. 5 illustrates a part of the cross section taken by cutting the hard member 62 on a cut surface passing through the third plane PL3 of FIG. 4. In addition, similar to FIG. 3, FIGS. 4 and 5 illustrate a state in which the raising base 13 is set to the inverted state.

As illustrated in FIG. 4 or 5, the rotation mechanism 14 is positioned in a lateral side of the housing groove 12 and is arranged inside the hollow 69 recessed toward the housing groove 12 (side wall surface 122) from the outer surface of the hard member 62.

In this hollow 69, a circular hole 691A penetrating one of the side wall surfaces 122 in the housing groove 12 is formed on the rotational axis RAx of a bottom 691 as illustrated in FIG. 4.

As illustrated in FIG. 4, a lid 693 is installed in an opening 692 of the hollow 69. This lid 693 has substantially the same planar shape as that of the opening 692 and is installed in the opening 692 to close the hollow 69. Furthermore, the lid 693 has a circular hole 693A penetrating from the front to the back thereof and extending along the rotational axis RAx.

As illustrated in FIG. 4 or 5, the rotation mechanism 14 has a bearing member 15, a shaft member 16, and a wire 17.

The bearing member 15 has an exterior shape substantially matching with an interior shape of the hollow 69, and is a member fitted (fixed) to the hollow 69 to rotatably support the shaft member 16.

In this bearing member 15, a hollow 151 recessed from the opening 692 side to the bottom 691 side is formed on the surface of the opening 692 side as illustrated in FIG. 4 or 5. A circular hole 152 communicating with the housing groove 12 through the circular hole 691A is formed on the bottom of the hollow 151. In addition, a circular frame-shaped shaft support portion 153 protruding from an edge of the circular hole 152 to the housing groove 12 side so as to be fitted to the circular hole 691A is formed on the bottom.

The shaft member 16 is a member supported rotatably about the rotational axis RAx with respect to the bearing member 15 and the lid 693 to rotate the raising base 13. As illustrated in FIG. 4 or 5, the shaft member 16 has a shaft member body 161 (FIG. 4), a bulging portion 162, and a protruding portion 163.

The shaft member body 161 has a circular column shape extending along the rotational axis RAx, and its one end is fitted (fixed) to the fixation hole 131A. In addition, the shaft member body 161 is inserted into the shaft support portion 153 and is supported rotatably about the rotational axis RAx along with the raising base 13.

The bulging portion 162 is provided in the other end of the shaft member body 161 and is a plate body bulging outward from the outer peripheral surface of the shaft member body 161 (or toward a direction away from the rotational axis RAx) to be positioned inside the hollow 151.

The protruding portion 163 has a circular column shape protruding from a bottom surface of the shaft member body 161 having the circular column shape, the bottom surface facing the lid 693, and extending along the rotational axis RAx. In addition, the protruding portion 163 is inserted through the circular hole 693A and is supported rotatably about the rotational axis RAx.

The wire 17 is routed inside the operating unit 7, the flexible pipe 64, the bendable portion 63, and the hard member 62 and has one end connected to the operating unit 7 and the other end connected to the bulging portion 162 inside the hollow 151. In addition, the wire 17 transmits force to the shaft member 16 according to a user operation on the operating unit 7 by a doctor or the like.

Specifically, when the wire 17 is pulled to the proximal end side in response to a user operation on the operating unit 7 by a doctor or the like, the shaft member 16 is rotated counterclockwise in FIG. 5 around the rotational axis RAx. With this, the raising base 13 is rotated along with the shaft member 16 and is rotated clockwise in FIG. 3 around the rotational axis RAx so as to be set in the raised state in which the raising base 13 is erected against the insertion axis Ax.

Meanwhile, when pulling of the wire 17 toward the proximal end side is released in response to a user operation on the operating unit 7 by a doctor or the like (the wire 17 is returned to the distal end side), the shaft member 16 is rotated clockwise in FIG. 5 around the rotational axis RAx until the bulging portion 162 abuts on the side wall surface 154 (FIG. 5) of the hollow 151. In addition, the raising base 13 is rotated counterclockwise in FIG. 3 around the rotational axis RAx along with the shaft member 16 until the bulging portion 162 abuts on the side wall surface 154. That is, the raising base 13 abuts on the bearing member 15 (side wall surface 154) in order to restrict further rotation of raising base 13 so that the raising base 13 is not further rotated counterclockwise in FIG. 3 to abut on the bottom 123. In addition, the raising base 13 is set to the inverted state (FIGS. 3 to 5) inverted from the raised state while the side wall surface 154 and the bulging portion 162 abut on each other. That is, the bearing member 15 functions as a rotation restricting member according to the present disclosure.

The ultrasonic endoscope 2 according to this embodiment described above has the following advantageous effects.

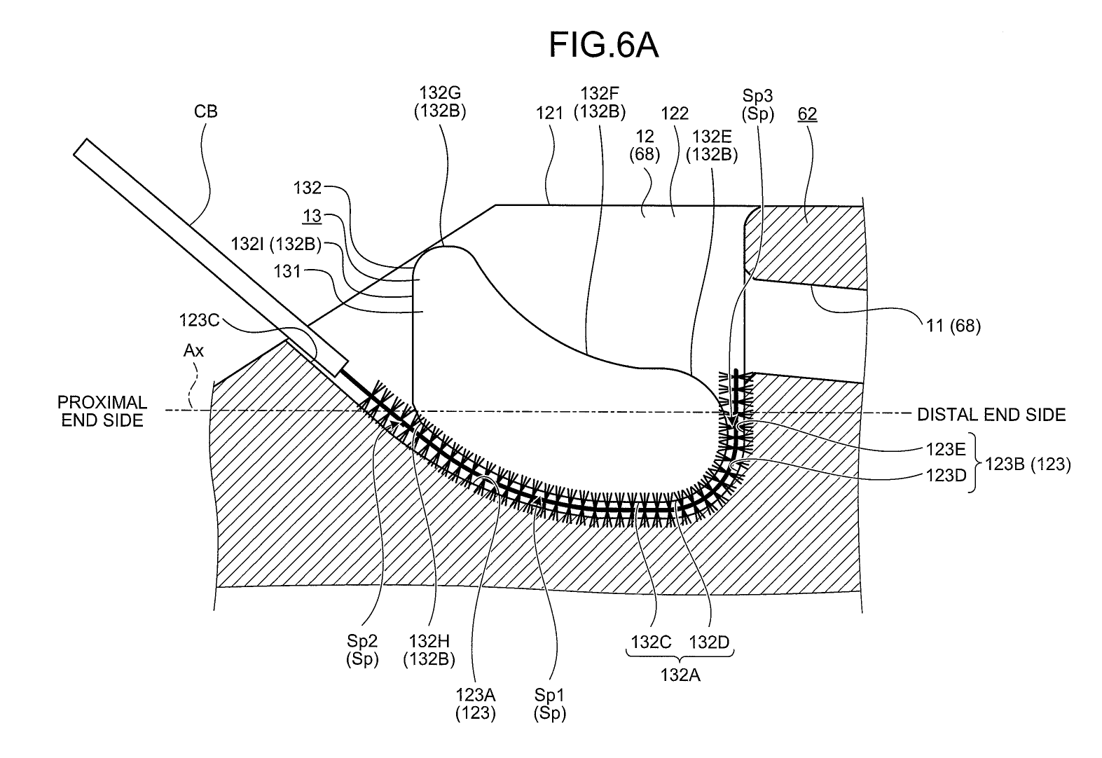

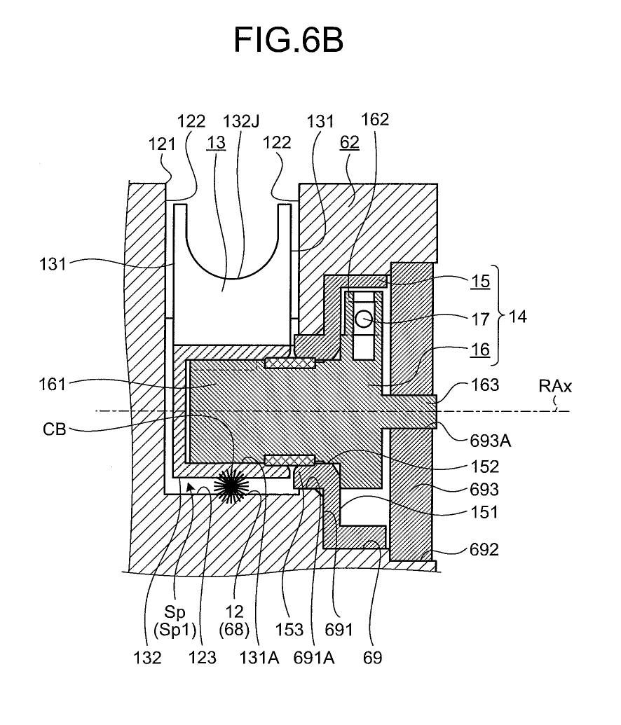

FIGS. 6A and 6B are diagrams illustrating advantageous effects of this embodiment. Specifically, FIG. 6A is a cross-sectional view corresponding to FIG. 3. FIG. 6B is a cross-sectional view corresponding to FIG. 4.

In the ultrasonic endoscope 2 according to this embodiment, a space Sp continuously extending from the distal end side to the proximal end side and having both ends opened in the distal end side and the proximal end side is formed between the bottom 123 and the raising base 13 while the raising base 13 is set to the inverted state. For this reason, as illustrated in FIGS. 6A and 6B, for example, if a cleaning brush CB is inserted into this space Sp from the distal end side, it is possible to penetrate the cleaning brush CB from the distal end side to the proximal end side of the space Sp. In addition, if the cleaning brush CB advances or recedes inside the space Sp, it is possible to perform brushing between the bottom 123 and the raising base 13 using the cleaning brush CB. That is, it is not necessary to sequentially change an insertion direction of the cleaning brush CB in order to perform cleaning between the bottom 123 and the raising base 13.

Therefore, using the ultrasonic endoscope 2 according to this embodiment, it is possible to simplify a cleaning work and reduce a work time of the cleaning.

In the ultrasonic endoscope 2 according to this embodiment, the space Sp includes a uniform portion Sp1 and an introduction portion Sp2. For this reason, for example, if a brush bristle of the cleaning brush CB has a diameter larger than the gap of the uniform portion Sp1, it is possible to collectively perform brushing from the distal end side to the proximal end side of the uniform portion Sp1 just by moving the cleaning brush CB forward or backward inside the space Sp. In addition, since the gap of the introduction portion Sp2 is larger than the gap of the uniform portion Sp1, it is possible to easily insert the cleaning brush CB from the distal end side or the proximal end side of the space Sp.

Meanwhile, if the wire 17 is directly connected to the raising base 13, the raising base 13 abuts on the bottom 123 when the raising base 13 is set to the inverted state. Therefore, it is difficult to form the space Sp having the uniform gap between the raising base 13 and the bottom 123.

In comparison, in the ultrasonic endoscope 2 according to this embodiment, the bulging portion 162 that bulges outward from the shaft member 16 abuts on the bearing member 15 (the side wall surface 154 (FIG. 5)) and thus restricts further rotation of the shaft member 16 (raising base 13) so as to set the raising base 13 to the inverted state. For this reason, it is possible to reliably form the space Sp by setting the raising base 13 to the inverted state.

In the ultrasonic endoscope 2 according to this embodiment, the space Sp has the curved surfaces provided in each of the outer peripheral surface 132 of the raising base 13 (including the first to third curved surfaces 132C to 132E, and the sixth curved surface 132H) and the bottom 123 (including the distal end side bottom 123A, the proximal end side bottom 123B). In particular, the distal end side bottom 123A and the proximal end side bottom 123B are smoothly connected without any corner part. This similarly applies to the first to third curved surfaces 132C to 132E and the sixth curved surface 132H. For this reason, when the cleaning brush CB advances or recedes inside the space Sp, it is possible to perform brushing smoothly without any obstacle on the bottom 123 or the raising base 13.

In the ultrasonic endoscope 2 according to this embodiment, the sloped surface 123C is formed in the distal end side of the distal end side bottom 123A. For this reason, it is possible to easily insert the cleaning brush CB from the distal end side of the hard member 62 to the space Sp.

In particular, the ultrasonic endoscope 2 according to this embodiment is an oblique-viewing endoscope in which a subject is observed in a direction crossing the insertion axis Ax at an acute angle. If the aforementioned sloped surface 123C is provided in a side-viewing endoscope in which a subject is observed in a direction perpendicularly crossing the insertion axis Ax, the hard member 62 is lengthened in the direction of the insertion axis Ax. Therefore, by providing the sloped surface 123C in the oblique-viewing endoscope, it is possible to prevent the hard member 62 from lengthening in the insertion axis Ax and miniaturize the hard member 62.

### Other Embodiments

While a mode for embodying the present disclosure has been described hereinbefore, the present disclosure is not limited to the aforementioned embodiment.

Although the endoscope system 1 according to the aforementioned embodiment has both the function of creating an ultrasonographic image and the function of creating an endoscopic image, only any one of them may be provided in the endoscope system 1 without limiting thereto.

The aforementioned embodiment may be applied to the endoscope system 1 for observing the inside of the subject such as a mechanical building in an industrial field without limiting to the medical field.

Although the ultrasonic endoscope 2 of the aforementioned embodiment is an oblique-viewing endoscope in which a subject is observed in a direction crossing the insertion axis Ax at an acute angle, the ultrasonic endoscope 2 may be a side-viewing endoscope in which a subject is observed in a direction perpendicularly crossing the insertion axis Ax without limiting thereto.

In the aforementioned embodiment, the shapes of the distal end side bottom 123A, the proximal end side bottom 123B, the first to third curved surfaces 132C to 132E, and the sixth curved surface 132H are not limited to those described in the aforementioned embodiment, but other shapes may also be employed as long as the space Sp is formed continuously from the distal end side to the proximal end side, and both ends are opened in the distal end side and the proximal end side.

Using the endoscope according to the present disclosure, it is possible to simplify a cleaning work and reduce a work time for the cleaning.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope comprising:
an insertion portion that is insertable into a subject and allows a treatment tool to protrude from a distal end side of the insertion portion, the insertion portion being cleanable with a cleaning tool having a predetermined dimension, wherein
the insertion portion includes:
a hard member having
a treatment tool insertion hole through which the treatment tool is allowed to be inserted, and
a housing groove that communicates with the treatment tool insertion hole and extends toward a distal end from the treatment tool insertion hole; and
a raising base that is housed rotatably in the housing groove, wherein the raising base is set to be in a raised state in which the raising base is raised with respect to an insertion axis along which the insertion portion is inserted into the subject and in an inverted state in which the raising base is inverted, by rotating the raising base, and wherein the raising base is abuttable on the treatment tool inserted into the housing groove through the treatment tool insertion hole so that a protruding direction of the treatment tool from the housing groove is adjusted,
wherein
a first introduction portion, a uniform portion, and a second introduction portion are provided continuously to form a space from a distal end side to a proximal end side of the insertion portion and between the raising base and a bottom of the housing groove while the raising base is set to be in the inverted state,
the uniform portion having a first gap having a first distance smaller than the predetermined dimension of the cleaning tool between the bottom of the housing groove and the raising base,
the first introduction portion having a second gap having a second distance greater than the first distance and being positioned on the distal end side with respect to the uniform portion, and
the second introduction portion having a third gap having a third distance greater than the first distance and being positioned on the proximal end side with respect to the uniform portion.

2. The endoscope according to claim 1,
wherein
the raising base is raisable upward along a direction perpendicular to the insertion axis,
the raising base includes a lower outer circumferential surface, and
wherein
a rotatable center of the raising base is deviated upward with respect to the bottom of the housing groove by summation of the first distance and a radius curvature of the lower outer circumferential surface while the raising base is set to be in the inverted state.

3. The endoscope according to claim 1, further comprising:
a shaft member that is connected to and rotatably supports the raising base;
a wire that is connected at one end thereof to the shaft member and transmits force applied to the other end of the wire by a user to the shaft member so that the raising base is rotated via the shaft member; and
a rotation restricting member that abuts on the shaft member to restrict rotation of the raising base so that the raising base is set to be in the inverted state.

4. The endoscope according to claim 1, wherein
the space is defined by a lower surface of the raising base and the bottom of the housing groove, the lower surface and the bottom including at least one of a plane and a curved surface.

5. The endoscope according to claim 1, wherein
the insertion portion includes at a distal end side thereof a sloped surface sloped against a plane perpendicular to the insertion axis so that the insertion portion becomes thinner toward the distal end side.

6. The endoscope according to claim 5, further comprising
an optical observation unit for performing observation in a direction crossing the insertion axis at an acute angle.

7. A method of cleaning the insertion portion of the endoscope of claim 1, the method comprising:
setting the raising base to be in the inverted state;
inserting the brush having the predetermined dimension into the space between the raising base and the bottom of the housing groove, the space being open on a distal end and on a proximal end thereof, from the distal end or the proximal end; and
causing the brush to advance or recede within the space so that the space is cleaned end to end while the brush is kept there inside.
